(19) United States
(12) Patent Application Publication
(10) Pub. No.: US 2023/0149351 A1
(43) Pub. Date: May 18, 2023

(54) TREATMENT OF SYMPTOMATIC VIRAL DISEASES

(71) Applicant: SYNACT PHARMA ApS, Holte (DK)

(72) Inventor: Thomas Engelbrecht Nordkild JONASSEN, Holte (DK)

(21) Appl. No.: 17/906,375

(22) PCT Filed: Mar. 30, 2021
(86) PCT No.: PCT/EP2021/058240

§ 371 Date: Sep. 15, 2022

(30) Foreign Application Priority Data
Mar. 31, 2020 (EP) 20167256.5
Mar. 18, 2021 (EP) 21163417.5

(51) Int. Cl. A61K 31/402; A61P 31/14; A61K 45/06

(52) U.S. Cl. CPC A61K 31/402; A61K 45/06; A61P 31/14

(57) ABSTRACT

Provided is a composition comprising a phenyl pyrrole aminoguanidine derivative for use in a method of treating viral disorders and diseases, including symptomatic viral disorders and diseases, including symptomatic COVID-19.

### TECHNICAL FIELD

The present invention relates to a composition comprising a compound of formula (I), or (II), or a pharmaceutically acceptable derivative thereof, for use in a method of treating viral diseases and disorders, including symptomatic COVID-19.

### BACKGROUND

Historically, infectious diseases, particularly due to viruses, have been one of the most important contributors to human morbidity and mortality. They account for a large proportion of death and disability worldwide, and remain in certain regions the most important cause of ill health. The burden of viral diseases and disorders is illustrated by the recent outbreak of coronavirus disease 2019 (COVID-19).

COVID-19 is an infectious disease caused by the recently discovered coronavirus Severe Acute Respiratory Syndrome CoronaVirus 2 (SARS-CoV-2), often referred to as the COVID-19 virus. Viral airway infection COVID-19 is also referred to as 2019-nCoV acute respiratory disease (2019-nCoV ARD) and novel coronavirus pneumonia (NCP).

Most people infected with the COVID-19 virus will experience mild to moderate respiratory illness resembling mild pneumonia and recover without requiring special treatment. Older people, and those with underlying medical problems like cardiovascular disease, diabetes, chronic respiratory disease, and cancer are more likely to develop serious illness. Common symptoms include fever, tiredness and dry cough. Other symptoms include shortness of breath, aches and pains and sore throat.

The clinical spectrum of COVID-19 varies from asymptomatic or paucisymptomatic forms to clinical conditions characterized by respiratory failure that necessitates mechanical ventilation and intensive care unit support, to multiorgan and systemic manifestations in terms of sepsis, septic shock, and multiple organ dysfunction syndromes.

Severe disease present with dyspnoea, increased respiratory frequency, reduced blood oxygen saturation and/or lung infiltrates. Critical disease present with respiratory failure, septic shock, and/or multiple organ dysfunction (MOD) or failure (MOF).

Infection of the upper and lower respiratory tract with the COVID-19 virus can cause mild or highly acute respiratory syndrome with consequent release of pro-inflammatory cytokines, including interleukin IL-6 and IL-1β. The SARS-CoV-2 virus causes an increase in IL-6 and IL-1β, and elevated levels of cytokines is found to predict the severity of COVID-19.

Initially, there were no specific vaccines or treatments for COVID-19. Some treatments to date have shown some efficacy. Often, treatment is symptomatic, and oxygen therapy represents the major treatment intervention for patients with severe infection. Strategies for addressing respiratory failure include protective mechanical ventilation and high-flow nasal oxygen (HFNO) or non-invasive ventilation (NIV).

Virally triggered ARDS (acute respiratory distress syndrome) is characterized by capillary damage and plasma leakage to the alveolar sacs, which disrupts the blood-air barrier and severely impairs blood oxygenation. This can occur directly as a result of viral damage, or indirectly by overactivation of the immune system that triggers the infiltration of immune cells such as neutrophils and macrophages into the lung along with a “cytokine storm” - the excessive or uncontrolled production of cytokines such as TNF, inter-leukin (IL)-1b, IL-6, IL-12, and IFNc, and chemokines such as IL-8, MCP-1, and IP-10. This is, in principle, a protective response to limit virus spread but ends up doing more harm than good. Although some of the details may differ, cytokine storms are a common complication of respiratory infections caused by influenza A, SARS-CoV, MERS-CoV and SARS-CoV-2 viruses.

The melanocortin system is a set of neuropeptidergic and immunoendocrine signaling pathways that play an integral role in the homeostatic control of a diverse array of physiological functions, including melanogenesis, stress response, inflammation, immunomodulation and adrenocortical steroidogenesis. It consists of multiple components, including the five G protein-coupled melanocortin receptors: melanocortin receptor 1 (MC1R) to MC5R; peptide ligands: α, β, γ-melanocyte stimulating hormone (α, β, γ- MSH), adrenocorticotropic hormone (ACTH) secreted by the anterior pituitary; and endogenous antagonists. The biological functions of melanocortin system are mediated by the five melanocortin receptors (MCRs), which have distinct tissue distribution, convey different signalling and exert varying biological activities in different organ systems.

Phenyl pyrrole aminoguanidine derivatives with activity on the melanocortin receptors are disclosed in WO 2007/141343. One example of such compound is the anti-inflammatory AP1189 ((E)-N-trans-{3-[1-(2-nitrophenyl)-1H-pyrrol-2-yl]-allylidene}-aminoguanidium acetate) which was first shown to bind the MC1R (WO 2007/141343) and later was identified as a biased dual agonist at receptors MC1R and MC3R that does not provoke canonical cAMP generation. The mechanism of action of AP1189 is to promote resolution of inflammation through melanocortin receptor activation directly on macrophages, thereby reducing the pro-inflammatory activity of macrophages and stimulating macrophage efferocytosis, a specific ability to clear inflammatory cells (Montero-Melendez et al. 2015). This effect has shown to be effective in disease models of inflammatory and autoimmune diseases and the clinical potential of the approach is currently tested in a clinical phase 2 study in patients with active Rheumatoid Arthritis

### SUMMARY

The present inventors have found that the phenyl pyrrole aminoguanidine derivative AP1189 ((E)-N-trans-{3-[1-(2-nitrophenyl)-1H-pyrrol-2-yl]-allylidene}-aminoguanidium acetate) reduces the levels of pro-inflammatory cytokines IL-1 and IL-1β, and reduces neutrophil accumulation, as compared to vehicle. This implies that AP1189 and related compounds are candidates for the treatment of symptomatic COVID-19.

Samples from patients with COVID-19 pneumonia have shown that macrophages make up to 80% of total cells in the lung alveoli and play a key role for the hyperinflammation associated with the devastating effects of COVID-19 infections.

Clinical trial data has now demonstrated that in patients with COVID-19 induced pulmonary insufficiency receiving AP1189, none of them developed a need for more intensive pulmonary support and patients were discharged between day 3 and 9 of treatment.

AP1189 and related compounds are thus promising candidates for the treatment of viral diseases and disorders, via its capabilities to control or dampen inflammation, including the inopportune over-activation of the immune system and resulting hyper-inflammation, while rendering the immune system fully capable of fighting the underlying viral infection, in some instances even stimulating the immune system. This is called resolution therapy.

It is an aspect of the present disclosure to provide a composition comprising a compound of formula (I):

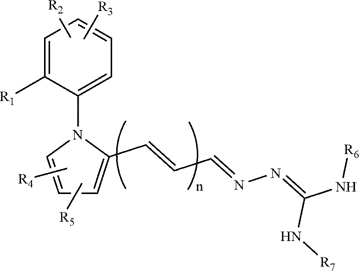

including tautomeric and stereoisomeric forms thereof; wherein n is 1; and R1 is CF3, CCl3, F, Cl, NO2 or CN, and R2, R3, R4, R5, R6, and R7 are hydrogen; or a pharmaceutically acceptable derivative thereof, for use in the treatment of viral diseases and disorders, such as symptomatic viral diseases and disorders, such as symptomatic viral diseases and disorders with respiratory symptoms including respiratory failure and ARDS.

It is an aspect of the present disclosure to provide a composition comprising a compound of formula (I):

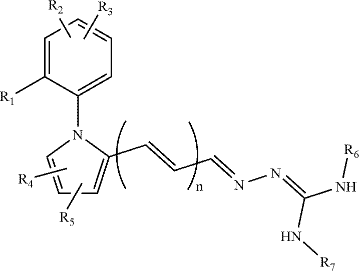

including tautomeric and stereoisomeric forms thereof; wherein n is 1; and R₁ is CF₃, CCl₃, F, Cl, NO₂ or CN, and R₂, R₃, R₄, R₅, R₆, and R₇ are hydrogen; or a pharmaceutically acceptable derivative thereof, for use in the treatment of symptomatic COVID-19, such as symptomatic COVID-19 with respiratory symptoms including respiratory failure and ARDS.

In one embodiment said compound is (E)-N-trans-{3-[1-(2-nitrophenyl)-1H-pyrrol-2-yl]-allylidene}-aminoguanidinium, and pharmaceutically acceptable salts thereof.

In one embodiment said compound is (E)-N-trans-{3-[1-(2-nitrophenyl)-1H-pyrrol-2-yl]-allylidene}-aminoguanidinium acetate (AP1189).

### DESCRIPTION OF DRAWINGS

Test Item A = AP1189 (E)-N-trans-{3-[1-(2-nitrophenyl)-1H-pyrrol-2-yl]-allylidene}-aminoguanidium acetate.

FIG. 1: Pre-treatment with AP1189 reduces the levels of the pro-inflammatory cytokines IL-1β compared to pretreatment with vehicle by 37%.

FIG. 2: Pre-treatment with AP1189 reduces the levels of the pro-inflammatory cytokines IL-6 compared to pretreatment with vehicle by 59%.

FIG. 3: Pre-treatment with AP1189 reduces neutrophil accumulation compared to pretreatment with vehicle by 70%.

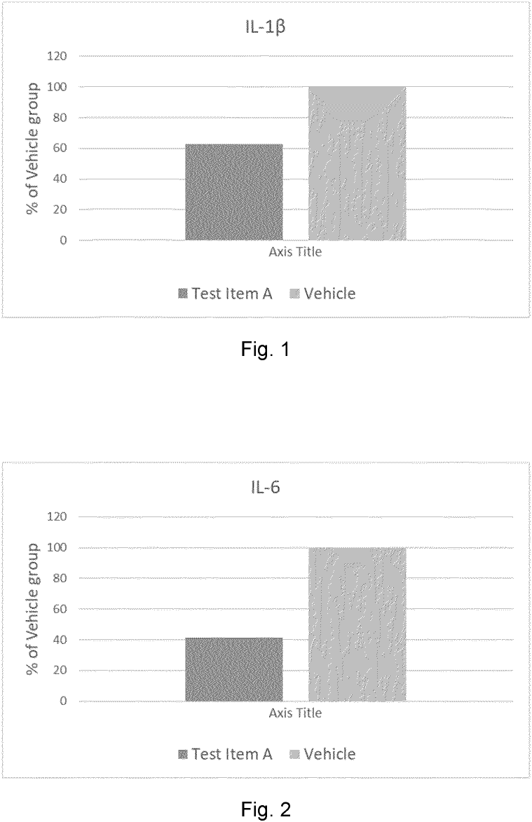

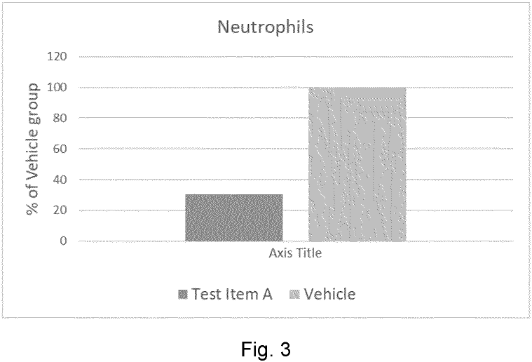

### DEFINITIONS

The term “pharmaceutically acceptable derivative” in the present context includes pharmaceutically acceptable salts, which indicate a salt which is not harmful to the patient. Such salts include pharmaceutically acceptable basic or acid addition salts as well as pharmaceutically acceptable metal salts, ammonium salts and alkylated ammonium salts. A pharmaceutically acceptable derivative further includes esters and prodrugs, or other precursors of a compound which may be biologically metabolized into the active compound, or crystal forms of a compound. In one embodiment a pharmaceutically acceptable derivative is a pharmaceutically acceptable salt.

The term “acid addition salt” is intended to include “pharmaceutically acceptable acid addition salt” which indicates salts which are not harmful to the patient. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 66, 2, (1977) which is incorporated herein by reference.

The term “therapeutically effective amount” of a compound as used herein refers to an amount sufficient to cure, alleviate, prevent, reduce the risk of, or partially arrest the clinical manifestations of a given disease or disorder and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The terms “treatment” and “treating” as used herein refer to the management and care of a patient for the purpose of combating a condition, disease or disorder. The term is intended to include the full spectrum of treatments for a given condition from which the patient is suffering. The patient to be treated is preferably a mammal, in particular a human being. Treatment of animals, such as mice, rats, dogs, cats, horses, cows, sheep and pigs, is, however, also within the scope of the present context. The patients to be treated can be of various ages.

### DETAILED DESCRIPTION

Melanocortin (MC) receptors (MC1R-MC5R), a family of class A G protein-coupled receptors (GPCRs), are attractive therapeutic targets for a number of conditions due to their wide distribution and diversity of physiological processes they regulate. MC1R regulates UV light-induced skin tanning and other immune responses because of its expression on leukocytes. MC2R regulates cortisol production on the adrenal glands, whereas MC5R plays a role on exocrine glands secretions. MC3R and MC4R exert non-redundant functions on energy homeostasis in addition to specific anti-inflammatory roles; whereas MC3R activation is particularly protective for joint inflammation such as arthritis, MC4R provides neuroprotection in brain inflammation. Accordingly, an array of pathological situations could be targeted with MCR-drugs including skin conditions, cardiovascular pathologies, joint inflammation, obesity and cachexia.

Peripheral MC1R and MC3R can be pharmacologically activated to induce anti-inflammation. The endogenous agonist a-melanocyte-stimulating hormone (αMSH), like other protective mediators, is released by immune cells to counterbalance proinflammatory signals, thus preventing excessive tissue damage. In line with the resolution of inflammation concept, therapeutics targeting MC1R and MC3R act by mimicking the body’s own protective resources and might be characterized by a lighter burden of side effects.

Shown to be effective in rheumatic diseases since the early 1950s, the use of corticotropin or adrenocorticotropin hormone (ACTH) declined when synthetic glucocorticoids became available. However, the discovery of an alternative anti-inflammatory mechanism for ACTH involving activation of peripheral MC receptors on immune cells has revived the interest in developing novel ACTH-like molecules with no steroidogenic effects for the treatment of joint diseases such as gout or RA (rheumatoid arthritis). However, the limitation in the translational delivery of novel MC drugs besides the marketed ACTH formulations is imposed by the lack of receptor selectivity achieved so far.

Innovative approaches in G protein-coupled receptor drug discovery might help to overcome this limitation. Allosteric modulation consists in the ability of a molecule to enhance (positive modulation) or reduce (negative modulation) the effect of the endogenous ligand by binding to a distinct site of the receptor protein, termed allosteric site. A higher degree of selectivity is expected as allosteric regions are less conserved among the five MCRs, and indeed, allosteric modulators at MC4R are currently under development for the treatment of obesity.

Another emerging concept of significant therapeutic interest is the one of biased agonism. The obsolete notion that receptors could exist in two unique conformations, the active one and the inactive one, has been replaced with the conception that multiple active conformations can exist, each one creating a distinct signal yielding multiple functional outcomes. Receptor activation, rather than linear and static, is emerging as a highly dynamic and multidimensional process in which a diversity of active conformations may be induced by different molecules leading to distinct effects.

The small molecule AP1189 ((E)-N-trans-{3-[1-(2-nitrophenyl)-1H-pyrrol-2-yl]-allylidene}-aminoguanidium acetate) has been characterized as a biased agonist at receptors MC1R and MC3R, which does not induce canonical cAMP generation, but cause ERK1/2 phosphorylation, a signaling responsible for the proefferocytic effect evoked in mouse primary macrophages. AP1189 was shown to reduce cytokine release in macrophages, whereas no melanogenesis was induced by AP1189 in melanocytes. In vivo, oral AP1189 elicits anti-inflammatory actions in peritonitis and accelerated the resolution phase, and afforded significant reduction of macroscopic and histological parameters of joint disruption in experimental inflammatory arthritis. AP1189 is thus a biased dual agonist at MC1R and MC3R with anti-inflammatory properties together with a lack of effect on melanogenesis.

The dysregulated release of cytokines has been identified as one of the key factors behind poorer outcomes in respiratory viral infections. This “cytokine storm” produces an excessive inflammatory and immune response, especially in the lungs, leading to acute respiratory distress (ARDS), pulmonary edema and multi-organ failure. Alleviating this inflammatory state is crucial to improve prognosis.

Patients with respiratory symptoms that require mechanical ventilation, such as pneumonia and ARDS, often present with local inflammation, which can spread into life-threatening systemic inflammation. It is an important task to control inflammation in these patients, to inhibit inflammation (such as an over-activated inflammatory cascade) without blocking it and at the same time ensure the immune system remain effective in combating the condition. AP1189 in such settings can make a difference at least in such patients as it inhibits inflammation while at the same time it stimulated the immune system, including the ability to clear the inflammation faster. This is called resolution therapy.

### Viral Diseases and Disorders

It is an aspect of the present disclosure to provide a composition comprising a compound of formula (I):

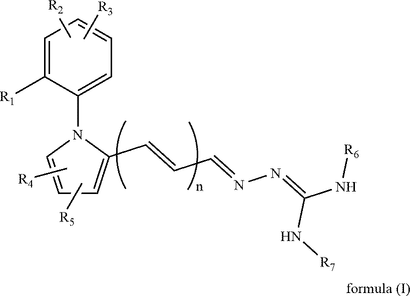

including tautomeric and stereoisomeric forms thereof; wherein n is 1; and R₁ is CF₃, CCl₃, F, Cl, NO₂ or CN, and R₂, R₃, R₄, R₅, R₆, and R₇ are hydrogen; or a pharmaceutically acceptable derivative thereof, for use in the treatment of viral diseases or disorders, such as for use in the treatment of symptomatic viral diseases or disorders.

It is also an aspect to provide the use of a composition comprising a compound of formula (I):

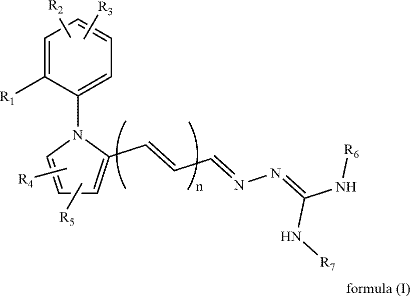

or a pharmaceutically acceptable derivative thereof, for the manufacture of a medicament for the treatment of viral diseases or disorders. such as for the manufacture of a medicament for the treatment of symptomatic viral diseases or disorders.

Also disclosed is a method for treating viral diseases or disorders, such as symptomatic viral diseases or disorders, said method comprising one or more steps of administration of a composition comprising a compound of formula (I):

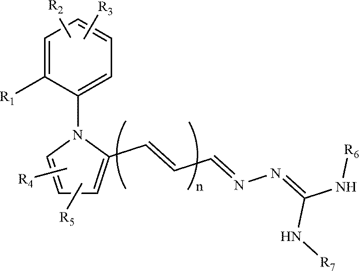

or a pharmaceutically acceptable derivative thereof, to an individual in need thereof.

In certain embodiments the composition for use according to the present disclosure comprises a compound of formula (II):

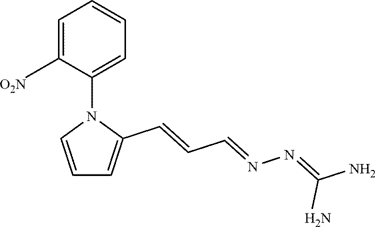

including tautomeric and stereoisomeric forms thereof; or a pharmaceutically acceptable derivative thereof, including pharmaceutically acceptable salts thereof.

In one embodiment said composition for use according to the present disclosure comprises a compound which is:
i) is an agonist of one or more MC receptors,
ii) is an agonist of the MC1R and MC3R, and/or
iii) is a biased agonist of the MC1R and MC3R.

Also disclosed is a composition for use according to the present disclosure comprising a compound selected from the group consisting of {3-[1-(2-nitrophenyl)-1H-pyrrol-2-yl]-allylidene}-aminoguanidine and (E)-N-trans-{3-[1-(2-nitrophenyl)-1H-pyrrol-2-yl]-allylidene}-aminoguanidine, or a pharmaceutically acceptable salt thereof, for use in the treatment of viral diseases or disorders, such as symptomatic viral diseases or disorders.

In one embodiment a pharmaceutically acceptable derivative thereof is a pharmaceutically acceptable salt of an inorganic acid or an organic acid.

In such embodiments an organic acid as referred to herein is selected from the group consisting of: formic acid, acetic acid, trichloroacetic acid, trifluoroacetic acid, propionic acid, benzoic acid, cinnamic acid, citric acid, fumaric acid, glycolic acid, lactic acid, maleic acid, malic acid, malonic acid, mandelic acid, oxalic acid, picric acid, pyruvic acid, salicylic acid, succinic acid, methanesulfonic acid, ethanesulfonic acid, tartaric acid, ascorbic acid, pamoic acid, bismethylene salicylic acid, ethanedisulfonic acid, gluconic acid, citraconic acid, aspartic acid, stearic acid, palmitic acid, EDTA, glycolic acid, p-aminobenzoic acid, glutamic acid, benzenesulfonic acid and p-toluenesulfonic acid.

In a particular embodiment said organic acid is acetic acid, succinic acid, tartaric acid or propionic acid; such as acetic acid.

In such embodiments an organic acid as referred to herein is selected from the group consisting of: hydrochloric acid, hydrobromic acid, hydroiodic acid, phosphoric acid, sulphuric acid and nitric acid.

Also disclosed is a composition comprising a compound selected from the group consisting of {3-[1-(2-nitrophenyl)-1H-pyrrol-2-yl]-allylidene}-aminoguanidinium acetate and (E)-N-trans-{3-[1-(2-nitrophenyl)-1H-pyrrol-2-yl]-allylidene}-aminoguanidinium acetate, for use in the treatment of viral diseases or disorders, such as symptomatic viral diseases or disorders.

In one embodiment the present disclosure provides a composition comprising (E)-N-trans-{3-[1-(2-nitrophenyl)-1H-pyrrol-2-yl]-allylidene}-aminoguanidinium or a pharmaceutically acceptable salt thereof for use in the treatment of viral diseases or disorders, such as symptomatic viral diseases or disorders.

In a particular embodiment the present disclosure provides a composition comprising (E)-N-trans-{3-[1-(2-nitrophenyl)-1H-pyrrol-2-yl]-allylidene}-aminoguanidinium acetate (AP1189) for use in the treatment of viral diseases or disorders, such as symptomatic viral diseases or disorders.

A viral disease or disorder according to the present disclosure is a disease or disorder which is caused by a viral infection, such as any symptom or down-stream effect of such viral infection.

It is an aspect to provide a composition comprising a compound of formula (I) or (II) as defined herein for use in the treatment of viral diseases or disorders, wherein said treatment of viral diseases or disorders include treatment, amelioration and/or alleviation of such viral diseases or disorders. It is understood that treatment, amelioration and/or alleviation of viral diseases or disorders include treatment, amelioration and/or alleviation of one or more symptoms of viral diseases or disorders.

In one embodiment there is provided a composition comprising a compound of formula (I) or (II) as defined herein for use in the treatment of one or more symptoms of viral diseases or disorders.

In one embodiment there is provided a composition comprising a compound of formula (I) or (II) as defined herein for use in the treatment of symptomatic viral diseases or disorders.

In one embodiment said one or more symptoms of viral diseases or disorders, or said symptomatic viral diseases or disorders, is inflammation, such as hyperinflammation.

In one embodiment said one or more symptoms of viral diseases or disorders, or said symptomatic viral diseases or disorders, is inflammation, such as hyperinflammation, in one or more organs. Inflammation in one or more organs may also be referred to as local inflammation.

In one embodiment said viral diseases or disorders, or said symptomatic viral diseases or disorders, present with inflammation, such as hyperinflammation.

In one embodiment there is provided a composition comprising a compound of formula (I) or (II) as defined herein for use in the treatment of virally induced hyperinflammation.

In one embodiment there is provided a composition comprising a compound of formula (I) or (II) as defined herein for use in the treatment of virally induced inflammatory conditions.

In one embodiment, hyperinflammation is characterized as C-reactive protein (CRP) >100 mg/l or ferritin 900 ng/ml.

In one embodiment said viral diseases or disorders, or said symptomatic viral diseases or disorders, present with inflammation, such as hyperinflammation, in one or more organs.

In one embodiment there is provided a composition comprising a compound of formula (I) or (II) as defined herein for use in the treatment of viral diseases or disorders associated with inflammation, such as hyperinflammation, in one or more organs.

In one embodiment said one or more organs are selected from the group consisting of lungs, the respiratory tract, kidney, liver, pancreas, spleen, exocrine glands, endocrine glands, lymph nodes, brain, heart, muscles, bone marrow, skin, skeleton, bladder, reproduction organs including the phallopian tubes, eye, ear, vascular system, the gastroinstestinal tract including small intestines, colon, rectum, canalis analis and the prostate gland.

In one embodiment there is provided a composition comprising a compound of formula (I) or (II) as defined herein for use in the treatment of end-organ damage in viral diseases or disorders.

In one embodiment there is provided a composition comprising a compound of formula (I) or (II) as defined herein for use in the treatment of inflammatory viral diseases or disorders.

In one embodiment there is provided a composition comprising a compound of formula (I) or (II) as defined herein for use in the treatment of viral diseases or disorders with local inflammation or systemic inflammation.

In one embodiment there is provided a composition comprising a compound of formula (I) or (II) as defined herein for use in the treatment of viral diseases or disorders with a local inflammatory condition.

In one embodiment there is provided a composition comprising a compound of formula (I) or (II) as defined herein for use in the treatment of viral diseases or disorders (such as symptomatic viral diseases or disorders) with a local inflammatory condition in one or more organs selected from the group consisting of lungs, the respiratory tract, kidney, liver, pancreas, spleen, exocrine glands, endocrine glands, lymph nodes, brain, heart, muscles, bone marrow, skin, skeleton, bladder, reproduction organs including the phallopian tubes, eye, ear, vascular system, the gastroinstestinal tract including small intestines, colon, rectum, canalis analis and the prostate gland.

It is noted that reference to viral diseases or disorders and reference to symptomatic viral diseases or disorders may be used interchangeably herein.

### Respiratory System

In one embodiment there is provided a composition comprising a compound of formula (I) or (II) as defined herein for use in the treatment of viral diseases or disorders which is a viral respiratory infection, such as a viral lower respiratory infection.

In one embodiment there is provided a composition comprising a compound of formula (I) or (II) as defined herein for use in the treatment of viral respiratory infections, such as viral lower respiratory infection.

In one embodiment viral diseases or disorders is a viral respiratory infection with impaired oxygenation, such as a viral lower respiratory infection with impaired oxygenation.

In one embodiment there is provided a composition comprising a compound of formula (I) or (II) as defined herein for use in the treatment of viral diseases or disorders of the lung.

In one embodiment there is provided a composition comprising a compound of formula (I) or (II) as defined herein for use in the treatment of viral respiratory diseases or disorders.

In one embodiment there is provided a composition comprising a compound of formula (I) or (II) as defined herein for use in the treatment of viral diseases or disorders with inflammation in the respiratory system, such as in the lungs and/or respiratory tract.

In one embodiment there is provided a composition comprising a compound of formula (I) or (II) as defined herein for use in the treatment of viral diseases or disorders with one or more respiratory symptoms.

In one embodiment viral diseases or disorders with one or more respiratory symptoms present with impaired oxygenation.

In one embodiment said one or more respiratory symptoms are selected from the group consisting of cough, dry cough, dyspnea, impaired oxygenation, respiratory illness, respiratory dysfunction, respiratory failure, respiratory syndrome and acute respiratory disease (ARD).

In one embodiment there is provided a composition comprising a compound of formula (I) or (II) as defined herein for use in the treatment of viral diseases or disorders, wherein said viral diseases or disorders is severe disease. Severe disease present with dyspnoea, increased respiratory frequency, reduced blood oxygen saturation and/or lung infiltrates.

In one embodiment there is provided a composition comprising a compound of formula (I) or (II) as defined herein for use in the treatment of viral diseases or disorders, wherein said viral diseases or disorders is critical disease. Critical disease present with respiratory failure, septic shock, and/or multiple organ dysfunction (MOD) or multiple organ failure (MOF).

In one embodiment there is provided a composition comprising a compound of formula (I) or (II) as defined herein for use in the treatment of viral diseases or disorders, wherein said viral diseases or disorders comprise viral pneumonia, including mild pneumonia, pneumonia, and pneumonia with abnormal findings.

In one embodiment there is provided a composition comprising a compound of formula (I) or (II) as defined herein for use in the treatment of viral pneumonia.

In one embodiment there is provided a composition comprising a compound of formula (I) or (II) as defined herein for use in the treatment of viral diseases or disorders, wherein said viral diseases or disorders comprise viral bronchiolitis.

In one embodiment there is provided a composition comprising a compound of formula (I) or (II) as defined herein for use in the treatment of viral diseases or disorders with respiratory failure.

Respiratory failure results from inadequate gas exchange by the respiratory system, meaning that the arterial oxygen, carbon dioxide or both cannot be kept at normal levels. A drop in the oxygen carried in blood is known as hypoxemia; a rise in arterial carbon dioxide levels is called hypercapnia. Respiratory failure is classified as either Type 1 or Type 2, based on whether there is a high carbon dioxide level, and can be either acute or chronic. The definition of respiratory failure in clinical trials usually includes increased respiratory rate, abnormal blood gases (hypoxemia, hypercapnia, or both), and evidence of increased work of breathing. Respiratory failure causes an altered mental status due to ischemia in the brain.

In one embodiment said respiratory failure is respiratory failure Type 1.

In one embodiment said respiratory failure is respiratory failure Type 2.

In one embodiment said respiratory failure is acute.

In one embodiment said respiratory failure is chronic.

In one embodiment said viral disease or disorder is acute respiratory distress syndrome (ARDS).

In one embodiment there is provided a composition comprising a compound of formula (I) or (II) as defined herein for use in the treatment of viral diseases or disorders with acute respiratory distress syndrome (ARDS).

In one embodiment there is provided a composition comprising a compound of formula (I) or (II) as defined herein for use in the treatment of viral acute respiratory distress syndrome (ARDS).

Acute respiratory distress syndrome (ARDS) is a type of respiratory failure characterized by rapid onset of widespread inflammation in the lungs. Symptoms include shortness of breath, rapid breathing, and bluish skin coloration. For those who survive, a decreased quality of life is common. The underlying mechanism involves diffuse injury to cells which form the barrier of the microscopic air sacs of the lungs, surfactant dysfunction, activation of the immune system, and dysfunction of the body’s regulation of blood clotting. In effect, ARDS impairs the lungs’ ability to exchange oxygen and carbon dioxide. The primary treatment involves mechanical ventilation together with treatments directed at the underlying cause.

Local inflammatory conditions such as acute respiratory distress syndrome can develop into a life-threatening condition with development of systemic inflammatory distress syndrome (SIDS) and sepsis. Development of SIDS is associated with high circulating levels of pro-inflammatory cytokines as IL-1β and IL-6.

In one embodiment there is provided a composition comprising a compound of formula (I) or (II) as defined herein for use in the treatment of viral diseases and disorders with acute respiratory distress syndrome (ARDS) and systemic inflammatory distress syndrome (SIDS).

In one embodiment there is provided a composition comprising a compound of formula (I) or (II) as defined herein for use in the treatment of viral diseases and disorders with systemic inflammatory distress syndrome (SIDS) and/or sepsis.

In one embodiment there is provided a composition comprising a compound of formula (I) or (II) as defined herein for use in the treatment of viral systemic inflammatory distress syndrome (SIDS) and/or viral sepsis.

In one embodiment there is provided a composition comprising a compound of formula (I) or (II) as defined herein for use in the treatment of viral diseases and disorders which require mechanical ventilation. In one embodiment said mechanical ventilation include protective mechanical ventilation, high-flow nasal oxygen (HFNO) and non-invasive ventilation (NIV).

In one embodiment there is provided a composition comprising a compound of formula (I) or (II) as defined herein for use in the treatment of viral diseases and disorders with pulmonary insufficiency.

In one embodiment there is provided a composition comprising a compound of formula (I) or (II) as defined herein for use in the treatment of viral induced pulmonary insufficiency, such as viral pulmonary insufficiency.

In one embodiment said pulmonary insufficiency is defined as a need for supplementary oxygen to maintain normal saturation.

In one embodiment said pulmonary insufficiency is defined as SaPO₂ lower than 93% on spontaneous respiration.

In one embodiment there is provided a composition comprising a compound of formula (I) or (II) as defined herein for use in the treatment of viral diseases or disorders, wherein said compound for use reduces the time to recovery.

In one embodiment said compound for use reduces the time to recovery, such as reduces the time to recovery by one or more days, such as by 7 days, such as by 6 days, such as by 5 days, such as by 4 days, such as by 3 days, such as by 2 days, such as by 1 day, such as by at least 1 day. In one embodiment said compound for use reduces the time to recovery by 1-2 days, such as by 2-3 days, such as by 3-4 days, such as by 4-5 days, such as by 5-6 days, such as by 6-7 days.

Reducing the time to recovery in one embodiment is reducing the median time to recovery. Reducing the time to recovery in one embodiment is reducing the time to full recovery.

In one embodiment there is provided a composition comprising a compound of formula (I) or (II) as defined herein for use in the treatment of viral diseases or disorders, wherein said compound for use promotes inflammatory resolution.

In one embodiment there is provided a composition comprising a compound of formula (I) or (II) as defined herein for use in the treatment of viral diseases or disorders, wherein said compound for use reduces the risk of development of severe inflammation.

In one embodiment there is provided a composition comprising a compound of formula (I) or (II) as defined herein for use in the treatment of viral diseases or disorders, wherein said compound for use reduces the risk of development of severe acute respiratory distress syndrome (ARDS).

In one embodiment there is provided a composition comprising a compound of formula (I) or (II) as defined herein for use in the treatment of viral diseases or disorders, wherein said compound for use reduces the requirement for supplementary oxygen to maintain normal saturation.

In one embodiment there is provided a composition comprising a compound of formula (I) or (II) as defined herein for use in the treatment of viral diseases or disorders, wherein said compound for use reduces the risk of developing a need for more intensive pulmonary support.

### General

In one embodiment there is provided a composition comprising a compound of formula (I) or (II) as defined herein for use in the treatment of viral diseases or disorders with elevated levels of one or more cytokines, such as one or more pro-inflammatory cytokines, such as IL-6 and/or IL-1β.

In one embodiment there is provided a composition comprising a compound of formula (I) or (II) as defined herein for use in the treatment of viral diseases or disorders with a cytokine storm (also called hypercytokinemia).

In one embodiment there is provided a composition comprising a compound of formula (I) or (II) as defined herein for use in the treatment of an infectious cytokine storm (hypercytokinemia), such as a virally induced cytokine storm (hypercytokinemia).

A cytokine storm is a physiological reaction in which the innate immune system causes an uncontrolled and excessive release of pro-inflammatory cytokines. Normally, cytokines are part of the body’s immune response to infection, but their sudden release in large quantities can cause multisystem organ failure and death. Cytokine storms can be caused by a number of infectious and non-infectious etiologies, especially viral respiratory infections.

In one embodiment there is provided a composition comprising a compound of formula (I) or (II) as defined herein for use in the treatment of multisystem organ failure caused by viral hypercytokinemia.

In one embodiment there is provided a composition comprising a compound of formula (I) or (II) as defined herein for use in the treatment of viral diseases or disorders with cytokine release syndrome (CRS). CRS is a form of systemic inflammatory response syndrome (SIRS) that can be triggered by a variety of factors such as infections and certain drugs.

### Other Organs

In one embodiment there is provided a composition comprising a compound of formula (I) or (II) as defined herein for use in the treatment of viral diseases or disorders with renal inflammation and/or renal dysfunction.

In one embodiment there is provided a composition comprising a compound of formula (I) or (II) as defined herein for use in the treatment of viral diseases or disorders with acute renal failure or chronic renal failure.

In one embodiment there is provided a composition comprising a compound of formula (I) or (II) as defined herein for use in the treatment of viral diseases or disorders with end-stage renal disease.

In one embodiment there is provided a composition comprising a compound of formula (I) or (II) as defined herein for use in the treatment of a viral kidney infection (pyelonephritis), viral urinary tract infection (UTI) and/or viral kidney inflammation (nephritis).

In one embodiment there is provided a composition comprising a compound of formula (I) or (II) as defined herein for use in the treatment of a viral bladder infection and/or viral bladder inflammation (cystitis).

In one embodiment viral disease or disorder is cystitis. In one embodiment viral disease or disorder is severe haemorrhagic cystitis. In one embodiment viral disease or disorder is urinary obstruction.

In one embodiment there is provided a composition comprising a compound of formula (I) or (II) as defined herein for use in the treatment of a viral liver infection and/or viral liver inflammation (hepatitis), such as acute hepatitis and chronic hepatitis.

In one embodiment there is provided a composition comprising a compound of formula (I) or (II) as defined herein for use in the treatment of a viral liver cirrhosis.

In one embodiment there is provided a composition comprising a compound of formula (I) or (II) as defined herein for use in the treatment of a viral pancreas infection and/or viral pancreas inflammation (pancreatitis).

In one embodiment there is provided a composition comprising a compound of formula (I) or (II) as defined herein for use in the treatment of a viral intestinal infection and/or viral intestinal inflammation, such as of the small intestine and/or of the large intestine.

In one embodiment said viral disease or disorder is colitis. In one embodiment said viral disease or disorder is enteritis.

In one embodiment there is provided a composition comprising a compound of formula (I) or (II) as defined herein for use in the treatment of a viral brain infection and/or viral brain inflammation.

In one embodiment said viral disease or disorder is encephalitis.

In one embodiment said viral disease or disorder is progressive multifocal leukoencephalopathy (PML).

In one embodiment said viral brain infection and/or viral brain inflammation is progressive multifocal leukoencephalopathy, a rare and often fatal viral disease characterized by progressive damage or inflammation of the white matter of the brain at multiple locations.

In one embodiment said viral brain infection and/or viral brain inflammation is inflammation of the white matter of the brain at one or multiple locations.

In one embodiment there is provided a composition comprising a compound of formula (I) or (II) as defined herein for use in the treatment of a viral eye infection and/or viral eye inflammation.

In one embodiment said viral disease or disorder is retinitis.

In one embodiment there is provided a composition comprising a compound of formula (I) or (II) as defined herein for use in the treatment of spleen enlargement/atrophy, in particular virally-induced spleen enlargement/atrophy.

In one embodiment there is provided a composition comprising a compound of formula (I) or (II) as defined herein for use in the treatment of diffuse lymphoid organ atrophy, in particular virally-induced diffuse lymphoid organ atrophy.

### Viral Infection

It is an aspect to provide a composition comprising a compound of formula (I) or (II) as defined herein for use in the treatment of viral diseases or disorders disorder which is caused by a viral infection.

In one embodiment there is provided a composition comprising a compound of formula (I) or (II) as defined herein for use in the treatment of viral diseases or disorders disorder which is caused by a viral infection selected from the group consisting of Severe Acute Respiratory Syndrome CoronaVirus 2 (SARS-CoV-2), often referred to as the COVID-19 virus; SARS-CoV, MERS-CoV, the dengue virus and influenza virus (including Type A, Type B and Type C).

In one embodiment there is provided a composition comprising a compound of formula (I) or (II) as defined herein for use in the treatment of acute respiratory distress syndrome (ARDS) or pneumonia caused by a viral infection selected from the group consisting of Severe Acute Respiratory Syndrome CoronaVirus 2 (SARS-CoV-2), often referred to as the COVID-19 virus; SARS-CoV, MERS-CoV, the dengue virus and influenza virus (including Type A, Type B and Type C).

It is an aspect to provide a composition comprising a compound of formula (I) or (II) as defined herein for use in the treatment of dengue fever.

Dengue fever is a mosquito-borne tropical disease caused by the dengue virus. Symptoms typically include a high fever, headache, vomiting, muscle and joint pains, and a characteristic skin rash. In a small proportion of cases, the disease develops into severe dengue, also known as dengue hemorrhagic fever, resulting in bleeding, low levels of blood platelets and blood plasma leakage, or into dengue shock syndrome, where dangerously low blood pressure occurs.

In one embodiment there is provided a composition comprising a compound of formula (I) or (II) as defined herein for use in the treatment of dengue hemorrhagic fever or dengue shock syndrome.

In one embodiment there is provided a composition comprising a compound of formula (I) or (II) as defined herein for use in the treatment of acute respiratory distress syndrome (ARDS) caused by dengue virus.

It is an aspect to provide a composition comprising a compound of formula (I) or (II) as defined herein for use in the treatment of viral pneumonia caused by dengue virus.

It is an aspect to provide a composition comprising a compound of formula (I) or (II) as defined herein for use in the treatment of influenza.

It is an aspect to provide a composition comprising a compound of formula (I) or (II) as defined herein for use in the treatment of viral diseases or disorders caused by influenza. It is an aspect to provide a composition comprising a compound of formula (I) or (II) as defined herein for use in the treatment of influenza virus diseases or disorders.

Influenza, commonly known as “the flu”, is an infectious disease caused by an influenza virus. Symptoms can range from mild to severe and commonly include: high fever, runny nose, sore throat, muscle and joint pain, headache, coughing, and feeling tired.

It is an aspect to provide a composition comprising a compound of formula (I) or (II) as defined herein for use in the treatment of viral pneumonia caused by influenza.

In one embodiment there is provided a composition comprising a compound of formula (I) or (II) as defined herein for use in the treatment of acute respiratory distress syndrome (ARDS) caused by influenza virus.

### Symptomatic COVID-19

It is an aspect of the present disclosure to provide a composition comprising a compound of formula (I):

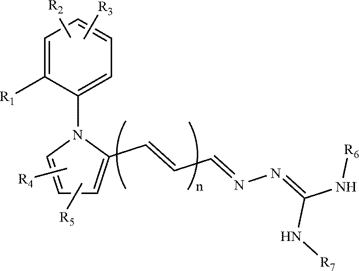

including tautomeric and stereoisomeric forms thereof; wherein n is 1; and R₁ is CF₃, CCl₃, F, Cl, NO₂ or CN, and R₂, R₃, R₄, R₅, R₆, and R₇ are hydrogen; or a pharmaceutically acceptable derivative thereof, for use in the treatment of symptomatic COVID-19.

It is also an aspect to provide the use of a composition comprising a compound of formula (I):

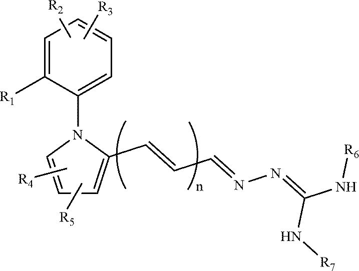

or a pharmaceutically acceptable derivative thereof, for the manufacture of a medicament for the treatment of symptomatic COVID-19.

Also disclosed is a method for treating symptomatic COVID-19, said method comprising one or more steps of administration of a composition comprising a compound of formula (I):

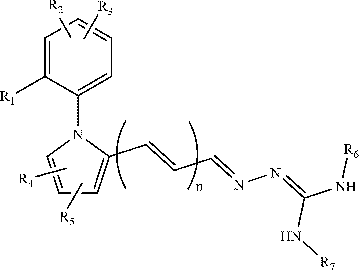

or a pharmaceutically acceptable derivative thereof, to an individual in need thereof.

In certain embodiments the composition for use according to the present disclosure comprises a compound of formula (II):

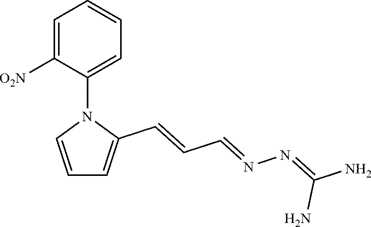 [SMILES: NC(N)=N/N=C/C=C/c1cccn1-c1ccccc1[N+](=O)[O-]]

including tautomeric and stereoisomeric forms thereof; or a pharmaceutically acceptable derivative thereof, for use in the treatment of symptomatic COVID-19.

In a preferred embodiment said composition for use according to the present disclosure comprises (E)-N-trans-{3-[1-(2-nitrophenyl)-1H-pyrrol-2-yl]-allylidene}-aminoguanidinium acetate (AP1189).

In one embodiment said composition for use according to the present disclosure comprises a compound which is:
i) is an agonist of one or more MC receptors,
ii) is an agonist of the MC1R and MC3R, and/or
iii) is a biased agonist of the MC1R and MC3R.

Also disclosed is a composition for use according to the present disclosure comprising a compound selected from the group consisting of {3-[1-(2-nitrophenyl)-1H-pyrrol-2-yl]-allylidene}-aminoguanidine and (E)-N-trans-{3-[1-(2-nitrophenyl)-1H-pyrrol-2-yl]-allylidene}-aminoguanidine, or a pharmaceutically acceptable salt thereof, for use in the treatment of symptomatic COVID-19.

In one embodiment a pharmaceutically acceptable derivative thereof is a pharmaceutically acceptable salt of an inorganic acid or an organic acid.

In such embodiments an organic acid as referred to herein is selected from the group consisting of: formic acid, acetic acid, trichloroacetic acid, trifluoroacetic acid, propionic acid, benzoic acid, cinnamic acid, citric acid, fumaric acid, glycolic acid, lactic acid, maleic acid, malic acid, malonic acid, mandelic acid, oxalic acid, picric acid, pyruvic acid, salicylic acid, succinic acid, methanesulfonic acid, ethanesulfonic acid, tartaric acid, ascorbic acid, pamoic acid, bismethylene salicylic acid, ethanedisulfonic acid, gluconic acid, citraconic acid, aspartic acid, stearic acid, palmitic acid, EDTA, glycolic acid, p-aminobenzoic acid, glutamic acid, benzenesulfonic acid and p-toluenesulfonic acid.

In a particular embodiment said organic acid is acetic acid, succinic acid, tartaric acid or propionic acid; such as acetic acid.

In such embodiments an organic acid as referred to herein is selected from the group consisting of: hydrochloric acid, hydrobromic acid, hydroiodic acid, phosphoric acid, sulphuric acid and nitric acid.

Also disclosed is a composition comprising a compound selected from the group consisting of {3-[1-(2-nitrophenyl)-1H-pyrrol-2-yl]-allylidene}-aminoguanidinium acetate and (E)-N-trans-{3-[1-(2-nitrophenyl)-1H-pyrrol-2-yl]-allylidene}-aminoguanidinium acetate, for use in the treatment of symptomatic COVID-19.

In one embodiment the present disclosure provides a composition comprising (E)-N-trans-{3-[1-(2-nitrophenyl)-1H-pyrrol-2-yl]-allylidene}-aminoguanidinium, or a pharmaceutically acceptable salt thereof, for use in the treatment of symptomatic COVID-19.

In a particular embodiment the present disclosure provides a composition comprising (E)-N-trans-{3-[1-(2-nitrophenyl)-1H-pyrrol-2-yl]-allylidene}-aminoguanidinium acetate for use in the treatment of symptomatic COVID-19.

In one embodiment said composition comprising a compound of formula (I) or (II) as defined herein is for use in a method of ameliorating and/or alleviating symptomatic COVID-19, such as ameliorating and/or alleviating one or more symptoms of COVID-19.

In one embodiment there is provided a composition comprising a compound of formula (I) or (II) as defined herein for use in the treatment of symptomatic COVID-19, wherein said symptomatic COVID-19 comprise one or more respiratory symptoms.

In one embodiment there is provided a composition comprising a compound of formula (I) or (II) as defined herein for use in the treatment of symptomatic COVID-19 with impaired oxygenation.

In one embodiment said one or more respiratory symptoms are selected from the group consisting of cough, dry cough, dyspnea, impaired oxygenation, respiratory illness, respiratory dysfunction, respiratory failure, respiratory syndrome and 2019-nCoV acute respiratory disease (2019-nCoV ARD).

In one embodiment there is provided a composition comprising a compound of formula (I) or (II) as defined herein for use in the treatment of symptomatic COVID-19, wherein said symptomatic COVID-19 is severe disease. Severe disease present with dyspnoea, increased respiratory frequency, reduced blood oxygen saturation and/or lung infiltrates.

In one embodiment there is provided a composition comprising a compound of formula (I) or (II) as defined herein for use in the treatment of symptomatic COVID-19, wherein said symptomatic COVID-19 is critical disease. Critical disease present with respiratory failure, septic shock, and/or multiple organ dysfunction (MOD) or failure (MOF).

In one embodiment there is provided a composition comprising a compound of formula (I) or (II) as defined herein for use in the treatment of symptomatic COVID-19, wherein said symptomatic COVID-19 comprise viral pneumonia, including mild pneumonia, pneumonia, pneumonia with abnormal findings and novel coronavirus pneumonia (NCP).

In one embodiment there is provided a composition comprising a compound of formula (I) or (II) as defined herein for use in the treatment of symptomatic COVID-19, wherein said symptomatic COVID-19 present with elevated levels of one or more cytokines, such as one or more pro-inflammatory cytokines, such as IL-6 and/or IL-1β.

In one embodiment there is provided a composition comprising a compound of formula (I) or (II) as defined herein for use in the treatment of symptomatic COVID-19 with respiratory failure.

Respiratory failure results from inadequate gas exchange by the respiratory system, meaning that the arterial oxygen, carbon dioxide or both cannot be kept at normal levels. A drop in the oxygen carried in blood is known as hypoxemia; a rise in arterial carbon dioxide levels is called hypercapnia. Respiratory failure is classified as either Type 1 or Type 2, based on whether there is a high carbon dioxide level, and can be either acute or chronic. The definition of respiratory failure in clinical trials usually includes increased respiratory rate, abnormal blood gases (hypoxemia, hypercapnia, or both), and evidence of increased work of breathing. Respiratory failure causes an altered mental status due to ischemia in the brain.

In one embodiment said respiratory failure is respiratory failure Type 1.

In one embodiment said respiratory failure is respiratory failure Type 2.

In one embodiment said respiratory failure is acute.

In one embodiment said respiratory failure is chronic.

In one embodiment there is provided a composition comprising a compound of formula (I) or (II) as defined herein for use in the treatment of symptomatic COVID-19 with acute respiratory distress syndrome (ARDS).

Acute respiratory distress syndrome (ARDS) is a type of respiratory failure characterized by rapid onset of widespread inflammation in the lungs. Symptoms include shortness of breath, rapid breathing, and bluish skin coloration. For those who survive, a decreased quality of life is common. The underlying mechanism involves diffuse injury to cells which form the barrier of the microscopic air sacs of the lungs, surfactant dysfunction, activation of the immune system, and dysfunction of the body’s regulation of blood clotting. In effect, ARDS impairs the lungs’ ability to exchange oxygen and carbon dioxide. The primary treatment involves mechanical ventilation together with treatments directed at the underlying cause.

Local inflammatory conditions such as acute respiratory distress syndrome can develop into a life-threatening condition with development of systemic inflammatory distress syndrome (SIDS) and sepsis. Development of SIDS is associated with high circulating levels of pro-inflammatory cytokines as IL-1β and IL-6.

In one embodiment there is provided a composition comprising a compound of formula (I) or (II) as defined herein for use in the treatment of symptomatic COVID-19 with acute respiratory distress syndrome (ARDS) and systemic inflammatory distress syndrome (SIDS).

In one embodiment there is provided a composition comprising a compound of formula (I) or (II) as defined herein for use in the treatment of symptomatic COVID-19 with systemic inflammatory distress syndrome (SIDS) and/or sepsis.

In one embodiment there is provided a composition comprising a compound of formula (I) or (II) as defined herein for use in the treatment of symptomatic COVID-19 which require mechanical ventilation. In one embodiment said mechanical ventilation include protective mechanical ventilation, high-flow nasal oxygen (HFNO) and non-invasive ventilation (NIV).

In one embodiment there is provided a composition comprising a compound of formula (I) or (II) as defined herein for use in the treatment of COVID-19 induced pulmonary insufficiency.

In one embodiment said COVID-19 induced pulmonary insufficiency is defined as a need for supplementary oxygen to maintain normal saturation.

In one embodiment said COVID-19 induced pulmonary insufficiency is defined as SaPO₂ lower than 93% on spontaneous respiration.

In one embodiment there is provided a composition comprising a compound of formula (I) or (II) as defined herein for use in the treatment of symptomatic COVID-19, wherein said compound for use reduces the time to recovery.

In one embodiment said compound for use reduces the time to recovery, such as reduces the time to recovery by one or more days, such as by 7 days, such as by 6 days, such as by 5 days, such as by 4 days, such as by 3 days, such as by 2 days, such as by 1 day, such as by at least 1 day. In one embodiment said compound for use reduces the time to recovery by 1-2 days, such as by 2-3 days, such as by 3-4 days, such as by 4-5 days, such as by 5-6 days, such as by 6-7 days.

Reducing the time to recovery in one embodiment is reducing the median time to recovery. Reducing the time to recovery in one embodiment is reducing the time to full recovery.

In one embodiment there is provided a composition comprising a compound of formula (I) or (II) as defined herein for use in the treatment of symptomatic COVID-19, wherein said compound for use promotes inflammatory resolution.

In one embodiment there is provided a composition comprising a compound of formula (I) or (II) as defined herein for use in the treatment of symptomatic COVID-19, wherein said compound for use reduces the risk of development of severe inflammation.

In one embodiment there is provided a composition comprising a compound of formula (I) or (II) as defined herein for use in the treatment of symptomatic COVID-19, wherein said compound for use reduces the risk of development of severe acute respiratory distress syndrome (ARDS).

In one embodiment there is provided a composition comprising a compound of formula (I) or (II) as defined herein for use in the treatment of symptomatic COVID-19, wherein said compound for use reduces the requirement for supplementary oxygen to maintain normal saturation.

In one embodiment there is provided a composition comprising a compound of formula (I) or (II) as defined herein for use in the treatment of symptomatic COVID-19, wherein said compound for use reduces the risk of developing a need for more intensive pulmonary support.

### Subject

The present compositions comprising a compound of formula (I) or (II) as defined herein is for use in the treatment of viral diseases or disorders as described herein in a subject in need thereof.

In some embodiments, the subject is infected with a virus.

In one embodiment the subject is a mammal. In particular the subject may be a human. In other embodiments the subject is an animal, such as a pet, including for example a cat, a dog, a rabbit, a horse, or a farm animal such as cattle or poultry, for example a cow, a bull, a sheep, a goat, a pig, a chicken, or a turkey.

In subjects with weakened immune system, such as HIV-positive subjects, a viral infection and symptomatic viral disorders or diseases can lead to potentially life-threatening multi-organ diseases.

Immunodeficiency can be the result of immunosuppressive agents routinely administered to subjects receiving a transplant, or it may be the result of a condition such as a genetic condition or an HIV infection.

In one embodiment there is provided a composition comprising a compound of formula (I) or (II) as defined herein for use in the treatment of viral diseases or disorders in a subject with an immunodeficiency.

In one embodiment said immunodeficiency is a T cell deficiency.

In one embodiment said immunodeficiency is a primary (or hereditary) immunodeficiency of T cells. These include some that cause complete insufficiency of T cells, such as severe combined immunodeficiency (SCID), Omenn syndrome, and Cartilage-hair hypoplasia.

In one embodiment said immunodeficiency is a secondary (or acquired) immunodeficiency of T cells. These include immunodeficiencies caused by HIV/AIDS, cancer chemotherapies, lymphoma, and glucocorticoid therapy.

In one embodiment the immune deficiency is complete or partial.

In some embodiments, the subject is HIV positive.

In one embodiment there is provided a composition comprising a compound of formula (I) or (II) as defined herein for use in the treatment of viral diseases or disorders in a subject from a high-risk population. These include HSCT patients (Hematopoietic stem-cell transplantation), SOT patients (solid organ transplant), elderly subjects and very young subjects.

In one embodiment elderly subjects are 65 years of age or above. In one embodiment elderly subjects are 70 years of age or above.

In one embodiment very young subjects are 1 year of age or below. In one embodiment very young subjects are 6 months of age or below. In one embodiment very young subjects are 3 months of age or below.

In one embodiment there is provided a composition comprising a compound of formula (I) or (II) as defined herein for use in the treatment of symptomatic COVID-19 in a subject selected from the group consisting of a subject from a high-risk population, such as a subject with an immunodeficiency, a HSCT patient (Hematopoietic stem-cell transplantation), a SOT patient (solid organ transplant), an elderly subject and a very young subject.

### Combination Therapies

In one embodiment the composition comprising a compound of formula (I) or (II), or a pharmaceutically acceptable derivative thereof as disclosed herein, comprises, separately or together, one or more additional active pharmaceutical ingredients.

In one embodiment said one or more additional active pharmaceutical ingredients are used for the treatment of a viral disease or disorder.

In one embodiment said one or more additional active pharmaceutical ingredients are used for the treatment of symptomatic COVID-19.

In one embodiment the composition comprising a compound of formula (I) or (II), or a pharmaceutically acceptable derivative thereof for use as disclosed herein is an add-on therapy to existing therapies.

In one embodiment the composition comprising a compound of formula (I) or (II), or a pharmaceutically acceptable derivative thereof for use as disclosed herein is an add-on therapy to one or more additional therapies used for the treatment of viral diseases or disorders.

In one embodiment said add-on therapy includes one or more of oxygen on nasal catheter and mechanical ventilation.

In one embodiment said symptomatic COVID-19 is also treated with oxygen on nasal catheter, such as oxygen on nasal catheter with flow between 2-5 LO₂/min, and/or and mechanical ventilation.

### Routes of Administration

It will be appreciated that the preferred route of administration will depend on the general condition and age of the subject to be treated, the nature of the condition to be treated, the location of the tissue to be treated in the body and the active ingredient chosen.

In one embodiment the composition comprising a compound of formula (I) or (II) as defined herein is administered by systemic administration, local administration, enteral administration or parenteral administration. Appropriate dosage forms for such administration may be prepared by conventional techniques.

### Systemic Administration

Systemic administration is capable of introducing the compound into the blood stream to ultimately target the sites of desired action.

Such routes of administration are any suitable routes, such as an enteral route, the oral, rectal, nasal, pulmonary, buccal, sublingual, transdermal, intracisternal, intraperitoneal, and parenteral (including subcutaneous, intramuscular, intrathecal, intravenous and intradermal) route.

In one embodiment, the composition comprising a compound of formula (I) or (II) as defined herein is administered by systemic administration.

### Oral Administration

Oral administration is normally for enteral drug delivery, wherein the compound is delivered through the enteral mucosa. Syrups and solid oral dosage forms, such as tablets, capsules and the like, are commonly used.

In one embodiment, the composition comprising a compound of formula (I) or (II) as defined herein is administered by oral administration.

### Parenteral Administration

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration, subcutaneous administration. Furthermore, parenteral administration includes inhalations and topical administration.

Accordingly, the compound may be administered topically to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth, and accordingly, parenteral administration may also include buccal, sublingual, nasal, rectal, vaginal and intraperitoneal administration as well as pulmonal and bronchial administration by inhalation or installation. Also, the compound may be administered topically to cross the skin.

The subcutaneous and intramuscular forms of parenteral administration are generally preferred.

### Local Treatment

The compound as disclosed herein is in one embodiment used as a local treatment, i.e. is introduced directly to the site(s) of action. Accordingly, the compound may be applied to the skin or mucosa directly, or the compound may be injected into the site of action, for example into the diseased tissue or to an end artery leading directly to the diseased tissue.

### Dosage

According to the present disclosure, the composition comprising a compound of formula (I) or (II) is administered to individuals in need of treatment in pharmaceutically effective doses. A therapeutically effective amount of a compound is an amount sufficient to cure, prevent, reduce the risk of, alleviate or partially arrest the clinical manifestations of a given disease and its complications. The amount that is effective for a particular therapeutic purpose will depend on the severity and the sort of the disorder as well as on the weight and general state of the subject. The compound may be administered one or several times per day, such as from 1 to 8 times per day, such as from 1 to 6 times per day, such as from 1 to 5 times per day, such as from 1 to 4 times per day, such as from 1 to 3 times per day, such as from 1 to 2 times per day, such as from 2 to 4 times per day, such as from 2 to 3 times per day. Alternatively, the compound may be administered less than once a day, for example once a day, such as once every second day, for example once every third day, such as once every fourth day, for example once every fifth day, such as once every sixth day, for example once every week.

In one embodiment the composition comprising a compound of formula (I) or (II) as defined herein is administered in a therapeutically effective amount, such as in an amount of 1 mg to 1000 mg compound of formula (I) or (II) per day.

It follows that in one embodiment the compound is administered in an amount of 1 mg to 1000 mg, such as 1 to 5 mg, 5 to 10 mg, 10 to 15 mg, 15 to 20 mg, 20 mg, 20 to 30 mg, 30 to 60 mg, 60 to 80 mg, 80 to 100 mg, 100 to 130 mg, 130 to 160 mg, 160 to 200 mg, 200 to 240 mg, 240 to 280 mg, 280 to 320 mg, 320 to 360 mg, 360 to 400 mg, 400 to 440 mg, 440 to 500 mg, 500 to 560 mg, 560 to 620 mg, 620 to 680 mg, 680 to 740 mg, 740 to 800 mg, 800 to 860 mg, 860 to 920 mg, 920 to 980 mg, 980 to 1000 mg, for example 500 to 1000 mg per day.

Per day means the dosage may be given in one dosage or divided in multiple dosages per day, including once a day (QD), twice a day (BID) and/or three times a day (TID).

In one embodiment the compound is administered in an amount of 100 mg once daily, 200 mg once daily, 300 mg once daily, 400 mg once daily, 500 mg once daily, 600 mg once daily, 700 mg once daily, 800 mg once daily, 900 mg once daily or 100 mg once daily.

In one embodiment the compound is administered in an amount of 100 mg once daily.

In one embodiment the compound is administered in an amount of 100 mg two times daily (BID) or 100 mg three times daily (TID).

In one embodiment the compound is administered in an amount of 200 mg two times daily (BID) or 200 mg three times daily (TID).

In one embodiment the compound is administered as a once daily oral dosing of 100 mg AP1189.

In another embodiment the compound is administered in an amount of 0.01 mg/kg bodyweight to 40 mg/ kg bodyweight, such as 0.01 mg/ kg bodyweight to 0.05 mg/ kg bodyweight, 0.05 to 0.1 mg/ kg bodyweight, 0.1 to 0.5 mg/ kg bodyweight, 0.5 mg to 1 mg/ kg bodyweight, 1 to 2 mg/ kg bodyweight, 2 to 3 mg/ kg bodyweight, 3 to 5 mg/ kg bodyweight, 5 to 10 mg/ kg bodyweight, 10 to 15 mg/ kg bodyweight, 15 to 20 mg/ kg bodyweight, 20 to 30 mg/ kg bodyweight, for example 30 to 40 mg/ kg bodyweight.

### Formulation

In one embodiment the composition comprising a compound of formula (I) or (II) as defined herein is a pharmaceutical composition, such as a pharmaceutically safe composition. The composition comprising a compound of formula (I) or (II)) as defined herein may be administered in any suitable way e.g. orally, sublingually, or parenterally, and it may be presented in any suitable form for such administration, e.g. in the form of solutions, suspension, aerosols, tablets, capsules, powders, syrups, implant or dispersions for injection.

In one embodiment, the composition comprising a compound of formula (I) or (II) as defined herein is formulated as a suspension.

In one embodiment, the composition comprising a compound of formula (I) or (II) as defined herein is formulated as an oral dose form, such as a solid oral dose form or pharmaceutical entity, such as tablets or capsules, or a liquid oral dose form. Methods for the preparation of solid pharmaceutical preparations are well known in the art.

In another embodiment the composition comprising a compound of formula (I) or (II) as defined herein is formulated as an injectable dose form.

In one embodiment the composition comprising a compound of formula (I) or (II) as defined herein is formulated in the form of a solid pharmaceutical entity, suitably as a tablet or a capsule

The compound (I) or (II) as the free base or the salt thereof may be administered alone or in combination with pharmaceutically acceptable carriers or excipients, in either single or multiple doses. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

### ITEMS

1. A composition comprising a compound of formula (I):

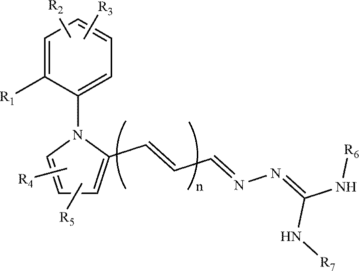

formula (I) including tautomeric and stereoisomeric forms thereof; wherein n is 1; and R₁ is CF₃, CCl₃, F, Cl, NO₂ or CN, and R₂, R₃, R₄, R₅, R₆, and R₇ are hydrogen; or a pharmaceutically acceptable derivative thereof, for use in the treatment of symptomatic COVID-19.

2. The composition for use according to item 1, wherein the compound is of formula (II):

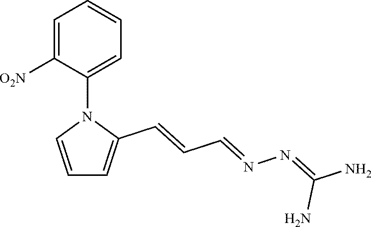 [SMILES: NC(N)=N/N=C/C=C/c1cccn1-c1ccccc1[N+](=O)[O-]]

including tautomeric and stereoisomeric forms thereof; or a pharmaceutically acceptable derivative thereof.

3. The composition for use according to any of the preceding items, wherein said compound is selected from the group consisting of {3-[1-(2-nitrophenyl)-1H-pyrrol-2-yl]-allylidene}-aminoguanidine and (E)-N-trans-{3-[1-(2-nitrophenyl)-1H-pyrrol-2-yl]-allylidene}-aminoguanidine, or a pharmaceutically acceptable salt thereof.

4. The composition for use according to any of the preceding items, wherein said pharmaceutically acceptable derivative thereof is a pharmaceutically acceptable salt of an inorganic acid or an organic acid.

5. The composition for use according to item 4, wherein said organic acid is selected from the group consisting of: formic acid, acetic acid, trichloroacetic acid, trifluoroacetic acid, propionic acid, benzoic acid, cinnamic acid, citric acid, fumaric acid, glycolic acid, lactic acid, maleic acid, malic acid, malonic acid, mandelic acid, oxalic acid, picric acid, pyruvic acid, salicylic acid, succinic acid, methanesulfonic acid, ethanesulfonic acid, tartaric acid, ascorbic acid, pamoic acid, bismethylene salicylic acid, ethanedisulfonic acid, gluconic acid, citraconic acid, aspartic acid, stearic acid, palmitic acid, EDTA, glycolic acid, p-aminobenzoic acid, glutamic acid, benzenesulfonic acid and p-toluenesulfonic acid.

6. The composition for use according to any items 4-5, wherein said organic acid is acetic acid, succinic acid, tartaric acid or propionic acid.

7. The composition for use according to item 4, wherein said inorganic acid is selected from the group consisting of: hydrochloric acid, hydrobromic acid, hydroiodic acid, phosphoric acid, sulphuric acid and nitric acid.

8. The composition for use according to item 6, wherein said organic acid is acetic acid.

9. The composition for use according to any of the preceding items, wherein said compound is selected from the group consisting of {3-[1-(2-nitrophenyl)-1H-pyrrol-2-yl]-allylidene}-aminoguanidinium acetate and (E)-N-trans-{3-[1-(2-nitrophenyl)-1H-pyrrol-2-yl]-allylidene}-aminoguanidinium acetate.

10. The composition for use according to any of the preceding items, wherein said compound is (E)-N-trans-{3-[1-(2-nitrophenyl)-1H-pyrrol-2-yl]-allylidene}-aminoguanidinium acetate (AP1189).

11. The composition for use according to any of the preceding items, wherein said compound:
i) is an agonist of one or more MC receptors,
ii) is an agonist of the MC1R and MC3R, and/or
iii) is a biased agonist of the MC1R and MC3R.

12. The composition for use according to any of the preceding items, wherein said symptomatic COVID-19 comprise one or more respiratory symptoms.

13. The composition for use according to any of the preceding items, wherein said symptomatic COVID-19 comprise one or more respiratory symptoms selected from the group consisting of cough, dry cough, dyspnea, respiratory illness, respiratory dysfunction, respiratory failure, respiratory syndrome and 2019-nCoV acute respiratory disease (2019-nCoV ARD).

14. The composition for use according to any of the preceding items, wherein said symptomatic COVID-19 comprise viral pneumonia, including mild pneumonia, pneumonia, pneumonia with abnormal findings and novel coronavirus pneumonia (NCP).

15. The composition for use according to any of the preceding items, wherein said symptomatic COVID-19 is severe disease.

16. The composition for use according to any of the preceding items, wherein said symptomatic COVID-19 is critical disease.

17. The composition for use according to any of the preceding items, wherein said symptomatic COVID-19 present with elevated levels of one or more pro-inflammatory cytokines, such as IL-6 and/or IL-1β.

18. The composition for use according to any of the preceding items, wherein said symptomatic COVID-19 is COVID-19 with respiratory failure.

19. The composition for use according to any of the preceding items, wherein said symptomatic COVID-19 is COVID-19 with acute respiratory distress syndrome (ARDS).

20. The composition for use according to any of the preceding items, wherein said symptomatic COVID-19 is COVID-19 with systemic inflammatory distress syndrome (SIDS).

21. The composition for use according to any of the preceding items, wherein said symptomatic COVID-19 require mechanical ventilation.

22. The composition for use according to any of the preceding items, wherein said symptomatic COVID-19 is COVID-19 induced pulmonary insufficiency.

23. The composition for use according to any of the preceding items, wherein said COVID-19 induced pulmonary insufficiency is defined as a need for supplementary oxygen to maintain normal saturation.

24. The composition for use according to any of the preceding items, wherein said COVID-19 induced pulmonary insufficiency is defined as SaPO2 lower than 93% on spontaneous respiration.

25. The composition for use according to any of the preceding items, wherein said compound for use reduces the time to recovery.

26. The composition for use according to any of the preceding items, wherein said compound for use promotes inflammatory resolution.

27. The composition for use according to any of the preceding items, wherein said compound for use reduce the risk of development of severe inflammation.

28. The composition for use according to any of the preceding items, wherein said compound for use reduces the risk of development of severe acute respiratory distress syndrome (ARDS).

29. The composition for use according to any of the preceding items, wherein said compound for use reduces the requirement for supplementary oxygen to maintain normal saturation.

30. The composition for use according to any of the preceding items, wherein said compound for use reduces the risk of developing a need for more intensive pulmonary support.

31. The composition for use according to any of the preceding items, wherein said composition comprises, separately or together, one or more additional active pharmaceutical ingredients, such as one or more additional active pharmaceutical ingredients used for the treatment of symptomatic COVID-19.

32. The composition for use according to any of the preceding items, wherein said composition is an add-on therapy to existing therapies.

33. The composition for use according to any of the preceding items, wherein said symptomatic COVID-19 is treated with oxygen on nasal catheter, such as oxygen on nasal catheter with flow between 2-5 LO₂/min.

34. The composition for use according to any of the preceding items, wherein said compound is administered in an amount of 1 mg to 1000 mg per day, such as 1 to 5 mg, 5 to 10 mg, 10 to 15 mg, 15 to 20 mg, 20 to 30 mg, 30 to 60 mg, 60 to 80 mg, 80 to 100 mg, 100 to 130 mg, 130 to 160 mg, 160 to 200 mg, 200 to 240 mg, 240 to 280 mg, 280 to 320 mg, 320 to 360 mg, 360 to 400 mg, 400 to 440 mg, 440 to 500 mg, 500 to 560 mg, 560 to 620 mg, 620 to 680 mg, 680 to 740 mg, 740 to 800 mg, 800 to 860 mg, 860 to 920 mg, 920 to 980 mg, 980 to 1000 mg, for example 500 to 1000 mg per day.

35. The composition for use according to any of the preceding items, wherein said compound is administered in an amount of 100 mg once daily, 200 mg once daily, 300 mg once daily, 400 mg once daily, 500 mg once daily, 600 mg once daily, 700 mg once daily, 800 mg once daily, 900 mg once daily or 100 mg once daily.

36. The composition for use according to any of the preceding items, wherein said compound is administered in an amount of 100 mg once daily.

37. The composition for use according to any of the preceding items, wherein said compound is administered in an amount of 100 mg two times daily (BID) or 100 mg three times daily (TID).

38. The composition for use according to any of the preceding items, wherein said compound is administered in an amount of 200 mg two times daily (BID) or 200 mg three times daily (TID).

39. The composition for use according to any of the preceding items, wherein said compound is administered as a once daily oral dosing of 100 mg AP1189.

40. The composition for use according to any of the preceding items, wherein said composition is pharmaceutically safe.

### EXAMPLES

### Example 1

Acute peritonitis like other local inflammatory conditions such as acute respiratory distress syndrome (ARDS) develop into a life-threatening condition with development of systemic inflammatory distress syndrome (SIDS) and sepsis. Development of SIDS is associated with high circulating levels of pro-inflammatory cytokines including IL-1β and IL-6.

Acute peritonitis was induced by the injection of 1 mg zymosan A (Sigma-Aldrich) i.p. in 0.5 ml sterile PBS in mice. Twelve hours later the mice were sacrificed by CO₂ exposure, and peritoneal cavities were washed with 4 ml ice-cold PBS containing 3 mM EDTA. Cells were stained with Turk’s solution (0.01% crystal violet in 3% acetic acid) and counted using a Neubauer hemocytometer or were stained with FITC-conjugated mAb for Ly-6G/Gr1, F4/80, and corresponding isotype controls (eBioscience, Hatfield, U.K.), and subjected to flow-cytometry analysis using a BD FACSCalibur platform (BD Biosciences, Oxford, U.K.).

Levels of IL-1β and IL-6 in quick frozen samples were quantified according to the manufacturer’s instructions using ELISA Ready-SET-Go! (eBioscience).

All animal studies were approved by and performed under the guidelines of the Ethical Committee for the Use of Animals, Barts and The London School of Medicine and Home Office regulations (Guidance on the Operation of Animals, Scientific Procedures Act, 1986). Male (7-8 wk old) C57BL/6J wild-type (WT) mice were purchased from Charles River Laboratories.

Test Item A = AP1189 (E)-N-trans-{3-[1-(2-nitrophenyl)-1 H-pyrrol-2-yl]-allylidene}-aminoguanidium acetate.

Test Item A, 1 mg/kg or vehicle treatment were given intraperitoneal 30 minutes before induction of the acute peritonitis. N=6 for in both groups.

### Results:

Pre-treatment with Test Item A reduced the levels of the pro-inflammatory cytokines IL-1β and IL-6 with 37% respectively 59% (p<0.05 vs vehicle) when compared to pretreatment with vehicle.

When compared to vehicle treatment neutrophil accumulation was reduced with 70% following pre-treatment with Test Item A.

### Example 2

SynAct has initiated dosing in part 2 of the clinical Phase II study with AP1189 in Covid-19 infected patients.

SynAct Pharma AB (“SynAct”) today announced that dosing in the second part of the exploratory clinical Phase 2 study with AP1189 in Covid-19 patients conducted under the RESOVIR collaboration has been initiated following completion of the initial open label part of study. The second part is a randomized double-blind placebo-controlled study in 54 [corrected from 56] Covid-19 patients at clinical sites at Universidade Federal de Minas, Belo Horizonte, Brazil.

The initial open label part of the study was conducted in 6 patients referred to hospital with Covid-19 induced pulmonary insufficiency, defined as a need for supplementary oxygen to maintain normal saturation. The patients, 4 women and 2 men aged between 38 and 59, all had SaPO2 lower than 93% on spontaneous respiration, and all 6 patients were treated with oxygen on nasal catheter with flow between 2-5 LO2/min. The patients were treated with once daily oral dosing of 100 mg AP1189 as add-on to standard therapy.

The compound was found to be safe and well tolerated, and the patients were discharged between day 3 and 9 of treatment as none of them developed a need for more intensive pulmonary support.

As no safety concerns have been identified, recruitment to part 2 of the study has been initiated. As of Mar. 16, 2021, 16 patients had been included to the second part of the study.

The second part of the study is set up to evaluate the safety and efficacy of a two-weeks dosing regimen with AP1189 vs placebo as add on therapy in patients with Covid-19 induced pulmonary insufficiency, defined as a need for supplementary oxygen to maintain normal saturation. Up to 54 patients will be randomized in a 2:1 ratio to receive AP1189 100 mg or placebo once daily, in addition to standard of care. The primary clinical objective of the study is to show reduction in time to respiratory recovery (i e time to normalization of oxygen saturation on ambient air). The top line results for the study are expected in Q2 2021.

There is an increasing need for effective treatments stopping the severe inflammation we see in the Covid-19 infected patients. We have started the second phase of the study to investigate whether AP1189 can promote inflammatory resolution and thereby reduce time to recovery and reduce the risk of development of severe ARDS.

Samples from patients with Covid-19 pneumonitis have shown that macrophages make up to 80% of total cells in the lung alveoli and play a key role for the hyperinflammation associated with the devastating effects of Covid-19 infections. As the AP189 specifically targets macrophages, we look very much forward to studying further the potential benefits provided by treatment of Covid-19 patients with AP1189.

Importantly, the study is not only aimed to test potential effect on clinical readouts as time to recovery, but also to investigate the effect of the compound on the activated inflammatory pathways in the Covid-19 infected patients. This work has been initiated with continued collection of samples from the patients and will be conducted in the laboratories in Belo Horizonte as well as in London as an integrated part of the RESOVIR collaboration.

### Example 3

The effect of treatment with AP1189 was tested in Covid-19 infected patients.

The study population consist of patients hospitalized with COVID-19 infection with impaired oxygenation. The study is conducted at sites in Belo Horizonte in Brazil.

The study consists of two parts:
Part 1: An open label study testing safety and tolerability of once daily oral dosing of AP1189 in six (6) hospitalized Covid-19 infected patients
Part 2: A randomized double-blind placebo controlled clinical trial testing once daily oral dosing of AP1189 or placebo in a 2:1 randomization in a total of 54 hospitalized Covid-19 infected patients. Part 2 was initiated following safety evaluation of part 1 as the compound was found safe and well tolerated by the studies safety monitoring board.

Dosing in the second part of the exploratory clinical Phase 2 study with AP1189 in Covid-19 patients has been initiated following completion of the initial open label part of study.

The study is dimensioned to identify a statistically significant treatment effect defined as a reduction in time to recovery relative to placebo treated controls with a significance level of 0.05 and a power of 80%, under the assumption that median time to full recovery is 11 days in the placebo treated patients, and the AP1189 treatment will reduce median time to recovery by 3 days.

Following a successful screening, consenting subjects who fulfil the enrolment criteria are and were in part A treated with once daily dosing of 100 mg AP1189 as add-on to any ongoing treatment. In part B patients will be randomized in a 2:1 ratio to either active or placebo:
Active Group (36 subjects): AP1189, dose 100 mg, once daily for 2 weeks (14 days) as an add-on to any ongoing treatment
Placebo Group (18 subjects): placebo once daily for 2 weeks (14 days) as an add-on to any ongoing treatment.

### Stopping Rules

In case of the occurrence of a serious medical event (e.g., stroke, convulsion etc.) or any SAE (serious adverse effect) considered related to the study drug, the investigator can decide to stop the study.

### Study Population

The study population consists of hospitalized subjects with COVID-19 infection and impaired oxygenation.

### 1.1. Subject Selection Criteria

The study can fulfil its objectives only if appropriate subjects are enrolled. The following eligibility criteria are designed to select subjects for whom protocol treatment is considered appropriate. All relevant medical and non-medical conditions should be taken into consideration when deciding whether this protocol is suitable for a subject.

### 1.2 Inclusion Criteria

Subject eligibility should be reviewed and documented by an appropriately qualified member of the investigator’s study team before subjects are included in the study. The following are requirements for entry into the study:
a. Written informed consent has been obtained prior to initiating any study-specific procedures
b. Male and female subjects, 18-85 years of age hospitalized with COVID-19 infection
c. Confirmed COVID-19 infection by the presence of SARS-CoV-2 nucleic acid by polymerase chain reaction (PCR)
d. Evidence of impaired oxygenation defined by SpO2 ≤93% on ambient air.
e. Duration of disease from first symptom <10 days before admission to hospital
f. Females of childbearing potential using reliable means of contraception or are postmenopausal (menstrual periods stopped at least 12 months ahead of the enrolment in the trial) or are surgically sterilized (the procedure must have been performed at least 6 months prior to screening)
g. Females of childbearing potential with a negative pregnancy
h. Test at screening and baseline.

### 1.3. Exclusion Criteria

Subjects meeting any of the following criteria are not eligible for participation in the study:
a. In the opinion of the investigator, progression to death is imminent and inevitable irrespective of the provision of treatment
b. Participating in other drug clinical trials (participation in COVID-19 antiviral trials where the compound is investigated is aimed to reduce the Virus infections. However, Remdesivir or dexamethasone in a dose not exceeding 10 mg/daily is permitted.
c. Any condition that in the view of the screening physician would suggest that the patient is unable to comply with study protocol and procedures (e.g., psychiatric disorders, dementia, patients in exclusive palliative care)
d. Subjects treated with immunosuppressive drugs (including microphenolate and cyclophosphamide)
e. HIV infection
f. Pregnant women or nursing (breastfeeding) mothers
g. Estimated glomerular filtration (eGFR) <30 ml/min
h. Severe liver dysfunction (Child-Pugh score C)
i. Medical history of per-oral glucocorticoid treatment (Exception is dexamethasone treatment not exceeding 10 mg/day related to the Covid-19 treatment)
j. Recruitment of Subjects

Potential subjects for this study will be identified amongst patients in the hospital. Potentially suitable subjects will be approached by the investigator at the site to ascertain whether they would be interested in participating in the study. Interested subjects will receive oral as well as written information about the study prior to consenting. No other study procedures will be performed prior to consenting.

### Results - Initial Open Label Part of the Study

6 patients referred to hospital with Covid-19 induced pulmonary insufficiency received once daily oral dosing of 100 mg AP1189 as add-on to standard therapy (oxygen on nasal catheter with flow between 2-5 LO2/min).

In these patients, the compound was found to be safe and well tolerated, and the patients were discharged between day 3 and 9 of treatment as none of them developed a need for more intensive pulmonary support. This indicated a potential of the compound to reduce the time to respiratory recovery (i.e. time to normalization of oxygen saturation on ambient air).

1-98. (canceled)

99. A method of treating a viral disease or disorder, said method comprising administering to a subject in need thereof a therapeutically effective amount of a composition comprising a compound of formula (I): 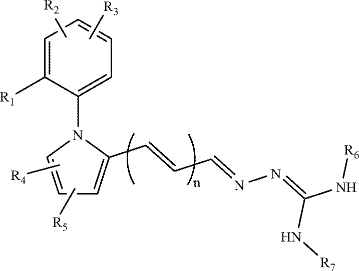
or a tautomeric or stereoisomeric form thereof; wherein:
n is 1,
R₁ is CF₃, CCl₃, F, Cl, NO₂ or CN, and
R₂, R₃, R₄, R₅, R₆, and R₇ are hydrogen;
or a pharmaceutically acceptable derivative thereof.

100. The method according to claim 99, wherein said compound is {3-[1-(2-nitrophenyl)-1 H-pyrrol-2-yl]-allylidene}-aminoguanidine or (E)-N-trans-{ 3-[1-(2-nitrophenyl)-1H-pyrrol-2-yl]-allylidene}-aminoguanidine, or a pharmaceutically acceptable salt thereof.

101. The method according to claim 99, wherein said pharmaceutically acceptable derivative thereof is a pharmaceutically acceptable salt thereof, such as a pharmaceutically acceptable salt of an inorganic acid or an organic acid; such as wherein said inorganic acid is hydrochloric acid, hydrobromic acid, hydroiodic acid, phosphoric acid, sulphuric acid or nitric acid; such as wherein said organic acid is formic acid, acetic acid, trichloroacetic acid, trifluoroacetic acid, propionic acid, benzoic acid, cinnamic acid, citric acid, fumaric acid, glycolic acid, lactic acid, maleic acid, malic acid, malonic acid, mandelic acid, oxalic acid, picric acid, pyruvic acid, salicylic acid, succinic acid, methanesulfonic acid, ethanesulfonic acid, tartaric acid, ascorbic acid, pamoic acid, bismethylene salicylic acid, ethanedisulfonic acid, gluconic acid, citraconic acid, aspartic acid, stearic acid, palmitic acid, EDTA, glycolic acid, p-aminobenzoic acid, glutamic acid, benzenesulfonic acid or p-toluenesulfonic acid.

102. The method according to claim 101, wherein said compound is {3-[1-(2-nitrophenyl)-1H-pyrrol-2-yl]-allylidene}-aminoguanidinium acetate or (E)-N-trans-{3-[1-(2-nitrophenyl)-1H-pyrrol-2-yl]-allylidene}-aminoguanidinium acetate (AP1189).

103. The method according to claim 99, wherein said compound is:
a) an agonist of one or more MC receptors,
b) an agonist of the MC1R and MC3R, and/or
c) a biased agonist of the MC1R and MC3R.

104. The method according to claim 99, wherein said viral disease or disorder is a symptomatic viral disease or disorder.

105. The method according to claim 104, wherein said symptomatic viral disease or disorder is symptomatic COVID-19, for example wherein said symptomatic COVID-19:
a) comprise one or more respiratory symptoms,
b) comprise one or more respiratory symptoms that is a cough, dry cough, dyspnea, respiratory illness, respiratory dysfunction, respiratory failure, respiratory syndrome or 2019-nCoV acute respiratory disease (2019-nCoV ARD),
c) comprise viral pneumonia, including mild pneumonia, pneumonia, pneumonia with abnormal findings or novel coronavirus pneumonia (NCP),
d) is severe disease,
e) is critical disease,
f) present with elevated levels of one or more pro-inflammatory cytokines, such as IL-6 and/or IL-1β,
g) is COVID-19 with respiratory failure,
h) is COVID-19 with acute respiratory distress syndrome (ARDS),
i) is COVID-19 with systemic inflammatory distress syndrome (SIDS),
j) require mechanical ventilation, or
k) is COVID-19 induced pulmonary insufficiency, such as said COVID-19 induced pulmonary insufficiency is defined as a need for supplementary oxygen to maintain normal saturation or wherein said COVID-19 induced pulmonary insufficiency is defined as SaPO2 lower than 93% on spontaneous respiration.

106. The method according to claim 99, wherein said viral disease or disorder is:
a) an inflammatory viral disease or disorder, such as a virally induced inflammatory condition, such as virally induced inflammation, such as virally induced hyperinflammation,
b) is virally induced hyperinflammation in one or more organs, such as wherein said hyperinflammation is characterized as C-reactive protein (CRP) >100 mg/l or ferritin 900 ng/ml, or
c) is a viral disease or disorder with local inflammation or with a local inflammatory condition, such as a local inflammatory condition in one or more organs,
such as wherein said one or more organs are a lung, the respiratory tract, kidney, liver, pancreas, spleen, exocrine gland, endocrine gland, lymph node, brain, heart, muscle, bone marrow, skin, skeleton, bladder, reproduction organ including the phallopian tubes, eye, ear, vascular system, the gastrointestinal tract including small intestine, colon, rectum, canalis analis or the prostate gland.

107. The method according to claim 99, wherein said viral disease or disorder is with:
a) inflammation in the respiratory system, such as in the lung and/or respiratory tract, or
b) one or more respiratory symptoms that are cough, dry cough, dyspnea, impaired oxygenation, respiratory illness, respiratory dysfunction, respiratory failure, respiratory syndrome or acute respiratory disease (ARD), such as wherein said respiratory symptom is respiratory failure type 1, respiratory failure type 2, acute respiratory failure or chronic respiratory failure.

108. The method according to claim 99, wherein said viral disease or disorder:
a) is acute respiratory distress syndrome (ARDS),
b) is severe disease, such as wherein said severe disease present with one or more of dyspnoea, increased respiratory frequency, reduced blood oxygen saturation and/or lung infiltrates,
c) is critical disease, such as wherein said critical disease present with one or more of respiratory failure, septic shock, and/or multiple organ dysfunction (MOD) or multiple organ failure (MOF).
d) is viral bronchiolitis,
e) is viral pneumonia, such as wherein said viral pneumonia is mild pneumonia, pneumonia, or pneumonia with abnormal findings,
f) is pulmonary insufficiency, such as wherein said pulmonary insufficiency is defined as a need for supplementary oxygen to maintain normal saturation, such as wherein said pulmonary insufficiency is defined as SaPO2 lower than 93% on spontaneous respiration,
g) requires mechanical ventilation, such as wherein said mechanical ventilation includes protective mechanical ventilation, high-flow nasal oxygen (HFNO) or non-invasive ventilation (NIV),
h) is a viral disease or disorder with systemic inflammation,
i) is systemic inflammatory distress syndrome (SIDS), or
j) is acute respiratory distress syndrome (ARDS) or systemic inflammatory distress syndrome (SIDS).

109. The method according to claim 99, wherein said treatment reduces the time to recovery.

110. The method according to claim 99, wherein said treatment:
a) promotes inflammatory resolution,
b) reduces the risk of development of severe inflammation,
c) reduces the risk of development of severe acute respiratory distress syndrome (ARDS),
d) reduces the requirement for supplementary oxygen to maintain normal saturation, and/or
e) reduces the risk of developing a need for more intensive pulmonary support.

111. The method according to claim 99, wherein the subject:
a) has elevated levels of one or more cytokines, such as one or more pro-inflammatory cytokines, such as IL-6 and/or IL-1β, and/or
b) is treated with oxygen on nasal catheter, such as oxygen on nasal catheter with flow between 2-5 LO2/min, and/or mechanical ventilation.

112. The method according to claim 99, wherein said viral disease or disorder is a viral disease or disorder with:
a) a cytokine storm (hypercytokinemia),
b) multisystem organ failure caused by viral hypercytokinemia,
c) cytokine release syndrome (CRS),
d) renal inflammation and/or renal dysfunction,
e) acute renal failure or chronic renal failure, or
f) end-stage renal disease,
such as wherein said viral disease or disorder is viral kidney infection (pyelonephritis), viral urinary tract infection (UTI) and/or viral kidney inflammation (nephritis).

113. The method according to claim 99, wherein said viral disease or disorder is a viral diseases or disorders with:
a) bladder infection and/or bladder inflammation (cystitis), such as severe haemorrhagic cystitis or urinary obstruction,
b) liver infection and/or liver inflammation (hepatitis), such as acute hepatitis and chronic hepatitis, and liver cirrhosis,
c) pancreas infection and/or pancreas inflammation (pancreatitis),
d) intestinal infection and/or intestinal inflammation, such as of the small intestine and/or of the large intestine, such as wherein said intestinal inflammation is colitis or enteritis,
e) brain infection and/or brain inflammation, such as wherein said brain inflammation is encephalitis; progressive multifocal leukoencephalopathy (PML); or inflammation of the white matter of the brain at one or multiple locations, or
f) eye infection and/or eye inflammation, such as wherein said eye inflammation is retinitis.

114. The method according to claim 99, wherein the viral disease or disorder is virally-induced:
a) spleen enlargement/atrophy, or
b) diffuse lymphoid organ atrophy.

115. The method according to claim 99, wherein said viral disease or disorder is caused by:
a) viral infection, such as a viral infection that is Severe Acute Respiratory Syndrome CoronaVirus 2 (SARS-CoV-2), SARS-CoV, MERS-CoV, the dengue virus or influenza virus (including Type A, Type B or Type C),
b) dengue virus, such as wherein said viral diseases or disorders is dengue hemorrhagic fever or dengue shock syndrome,
c) influenza virus, or
d) a virus that is SARS-CoV-2, SARS-CoV or MERS-CoV.

116. The method according to claim 99, wherein the treatment is in a subject:
a) with an immunodeficiency, such as wherein said subject with an immunodeficiency is a subject with a T cell deficiency, including a primary immunodeficiency of T cells such as severe combined immunodeficiency (SCID), Omenn syndrome, or Cartilage-hair hypoplasia, or a secondary immunodeficiency of T cells such as those caused by HIV/AIDS, cancer chemotherapies, lymphoma, or glucocorticoid therapy, and/or
b) from a high-risk population, including HSCT patients (Hematopoietic stem-cell transplantation), SOT patients (solid organ transplant), elderly subjects or very young subjects.

117. The method according to claim 99, wherein said composition:
a) comprises, separately or together, one or more additional active pharmaceutical ingredients, such as one or more additional active pharmaceutical ingredients used for the treatment of viral diseases or disorders, such as wherein said one or more additional active pharmaceutical ingredients are active pharmaceutical ingredients used for the treatment of symptomatic COVID-19,
b) is an add-on therapy to one or more additional therapies used for the treatment of viral diseases or disorders, and/or
c) is pharmaceutically safe.

118. The method according to claim 99, wherein said compound is administered:
a) in an amount of 1 mg to 1000 mg per day, such as 1 to 5 mg, 5 to 10 mg, 10 to 15 mg, 15 to 20 mg, 20 to 30 mg, 30 to 60 mg, 60 to 80 mg, 80 to 100 mg, 100 to 130 mg, 130 to 160 mg, 160 to 200 mg, 200 to 240 mg, 240 to 280 mg, 280 to 320 mg, 320 to 360 mg, 360 to 400 mg, 400 to 440 mg, 440 to 500 mg, 500 to 560 mg, 560 to 620 mg, 620 to 680 mg, 680 to 740 mg, 740 to 800 mg, 800 to 860 mg, 860 to 920 mg, 920 to 980 mg, 980 to 1000 mg, for example 500 to 1000 mg per day,
b) in an amount of 100 mg daily, 200 mg daily, 300 mg daily, 400 mg daily, 500 mg daily, 600 mg daily, 700 mg daily, 800 mg daily, 900 mg daily or 100 mg daily,
c) in an amount of 100 mg once daily, 100 mg two times daily (BID) or 100 mg three times daily (TID),
d) in an amount of 200 mg two times daily (BID) or 200 mg three times daily (TID), and/or
e) as a daily oral dosing.
